(19) United States
(12) Patent Application Publication
(10) Pub. No.: US 2010/0227333 A1
(43) Pub. Date: Sep. 9, 2010

(54) METHOD OF DETECTING INFECTION WITH UROGENITAL MYCOPLASMAS IN HUMANS AND A KIT FOR DIAGNOSING SAME

(76) Inventor: Shulamith Horowitz, Omer (IL)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/598,738

(22) PCT Filed: May 1, 2008
(86) PCT No.: PCT/IL2008/000584

§ 371 Date: Mar. 16, 2010

(30) Foreign Application Priority Data
May 3, 2007 (IL) 182958

(51) Int. Cl. G01N 33/53; C07K 14/195

(52) U.S. Cl. 435/7.1; 530/350

(57) ABSTRACT

A method and a kit are provided for detecting an infection by mycoplasma, particularly an urogenital infection in humans. The presence of specific anti-mycoplasma antibodies in a biological sample of a diagnosed subject is detected by reaction with an immobilized mixture of various antigenic determinants associated with a variety of pathological states.

### FIELD OF THE INVENTION

The present invention relates to detecting an infection by mycoplasma, particularly an urogenital infection in humans. A kit for diagnosing urogenital infection caused by a broad range of mycoplasma strains is provided.

### BACKGROUND OF THE INVENTION

Mycoplasmas are a group of unique bacteria that often cause silent infections in humans and animals and plants. In animals, they are notorious as causative agents of morbidity and mortality. In humans, a variety of disorders are associated with mycoplasma infections, an example being mycoplasmal pneumonia or common sexually transmitted infections whose precise diagnosing remains difficult [see, for example, Merck Manual, 17^{th} Ed. (1999) p. 1326]. There is an increasing amount of evidence accumulated in the literature regarding mycoplasmal pathogenic mechanisms, but well-established tests, that could significantly promote the analysis of urogenital mycoplasmal diseases, are not generally available. WO 07/033171 describes a possible detection of the mycoplasma presence in a sample based on the hydridization with RNA probes. WO 94/03810 describes a method of diagnosing a previous mycoplasma infection in a subject based on the effects of anti-mycoplasma antibodies from a patient's sample on the growth of standard mycoplasma culture. EP 278340 describes the use of Mycoplasma pneumoniae membrane antigens in diagnosing mycoplasmal infections. The lack of reliable and rapid diagnostic tests makes the identification of urogenital mycoplasma as the cause of disease very difficult, resulting in underestimation of their role in disease and therefore lack of diagnosis and appropriate treatment

Particularly, urogenital mycoplasmas have been shown to be involved in a variety of diseases and pathological states, including pregnancy complications (premature delivery, recurrent abortions), morbidity and mortality of the premature infant, infertility (mainly male factor), urethritis and prostatitis, reactive arthritis, and recently rheumatoid arthritis. The role of urogenital mycoplasmas in the development of human diseases has been emerging only in the last decade, and determining mycoplasmal serology has been difficult, due to lack of standardized antigen being further complicated by unavailability of a practical test in the market. The importance and significance of measuring anti-mycoplasma antibodies in diagnosing the infection and in predicting the risk of several human pathologies have been demonstrated also by the present inventors [see, for example, Horowitz S. et al: J. Rheumatol, 27 (2000) 2747-53].

In spite of various “home-made” techniques introduced in various countries, no efficient and reliable general diagnostic test has been available so far for detecting human urogenital mycoplasmas in clinical samples. As known to a person skilled in the art, the techniques that are available suffer from various drawbacks, including limited sensitivity and limited specificity (appearance of false-positive or false negative results), to name some. Therefore, it is an object of this invention to provide a generally applicable kit for the detection of urogenital infections caused by mycoplasmas.

It is another object of this invention to provide a method for detecting urogenital infections caused by specific mycoplasmas, by means of suitable antigens.

It is still another object of this invention to provide a method and a kit with a lowered number of false-positive and/or false negative results for diagnosing mycoplasmal urogenital infections.

It is still further object of this invention to provide a method and a kit that enables to differentiate between humans who are only colonized with mycoplasmas and do not develop disease and those who develop infection/infectious disease/post infection pathologies

Other objects and advantages of present invention will appear as description proceeds.

### SUMMARY OF THE INVENTION

The present invention provides a method of detecting infection with urogenital mycoplasma, by measuring the level of specific anti-mycoplasma antibodies in a biological sample taken from a patient to be diagnosed, comprising i) providing a plurality of biological specimens from a plurality of pathological states, in patients suffering from mycoplasmal urogenital infections; ii) preparing a whole cell preparation from said several mycoplasma strains obtained from patients; iii) providing a plurality of mycoplasmal antigenic determinants (from each species) to be used as the material for detection of specific antibodies in the patients' sample; iv) binding said mixed antigen on an immunoassay surface; v) binding of the patients' biological sample with said surface of step iv, thereby binding of anti-mycoplasma antibodies eventually present in said sample onto said surface; vi) contacting the surface with a reagent for detecting human antibodies. Thus, the determination of infection will be performed by measuring a wide spectrum (plurality) of specific antibodies to the same species. Said immunoassay surface means a protein-binding surface, used in the art, that usually comprises polymers having strong affinity for proteins, not releasing them even during washing steps; the polymers may comprise modified natural materials or synthetic materials. Said plurality of human strains preferably comprises at least six patients each infected with one or more mycoplasma species selected from the group consisting of Ureaplasma urealyticum, Ureaplasma parvum, Mycoplasma hominis, Mycoplasma fermentans, and Mycoplasma genitalium. In a preferred embodiment of the invention, said a concentrated pellet of mycoplasma cells, obtained by centrifugation from a mycoplasma culture (of each strain), is sonicated and the whole cell sonicate preparations from several strains are mixed (in equal amounts of proteins) to form the “mixed antigen” preparation. Essential is that each of the protein antigens comprising the mixture, is obtained from a strain isolated from a patient with a known pathology (i.e. urethritis, pregnancy complication, infertility, arthritis etc.) and that protein antigens are obtained from a plurality of pathologies, thereby providing the best possible representation of array of antigens expressed in disease. Said immunoassay surface may comprise an item selected from well plate and polymer sheet, or other means known in the art of the immunoassay design. Said reagents may comprise anti-human antibodies conjugated with an enzyme yielding a color or fluorescent reaction, and an enzyme substrate, or other means enabling the antibody visualization.

The invention is directed to a mixed mycoplasmal proteinaceous antigen obtained from a plurality of persons suffering from acute mycoplasmal genitourinary infection. The term proteinaceous relates to the antigenic composition that is obtained according to the invention, which comprises protein components derived from mycoplasma cells and which usually participate in eliciting the immune response against mycoplasma infections in the human body; said composition may comprise other components, (i.e. lipids) that may originate from the mycoplasma cells, and are usually presented as lipoproteins

The invention is directed to the use of a mixed antigen derived from a plurality of mycoplasma strains isolated from a plurality of patients afflicted with mycoplasma infections in detecting a urogenital infections, covering a broad range of mycoplasma species and strains.

The invention provides a method not only for the diagnosis of a genitourinary diseases associated with a mycoplasma infection in a human patient, but also quantifying the level of said antibody, comparing the level of said antibody in the patient's sample with standard samples of reference persons not suffering from acute mycoplasmal disease.

The invention relates to a novel kit for general use in detecting a urogenital infection in human, caused by mycoplasmas, eventually covering a broad range of strains and species, comprising i) a mixed antigen bound on a matrix, the mixed antigen originating from mycoplasmas isolated from a plurality of persons suffering from acute mycoplasma infections; ii) reagents for visualizing the presence of human antibodies; and iii) instructions for use. Said matrix may be a strip comprising a protein binding polymer, such as nitrocellulose or a suitable polymer, possibly comprising a sheet or a well for detecting the presence of human anti-mycoplasma antibodies. The kit may comprise, for example, a plurality of strips, each for characterizing one sample, or a device for parallel characterizing a plurality of samples. The samples may include a body fluids such as serum, or other. Said reagents are known in the art of immunoassays, and may comprise a conjugate of antihuman antibody with enzyme, enzyme substrate, etc.

The invention enables to follow the success of the treatments used for mycoplasmal urogenital infection in a patient in need of such treatment, by providing a method that comprises the reliable detection of the presence of an immune response to urogenital mycoplasma followed by administering to said patient an antibiotic known to be efficient. Said antibiotic may comprise, without being limited to it, azithromycin, tetracycline, erythromycine, and clarithromycin.

### BRIEF DESCRIPTION OF THE DRAWINGS

The above and other characteristics and advantages of the invention will be more readily apparent through the following examples, and with reference to the appended drawings, wherein:

FIG. 1. is a graph showing the antibody levels as determined in sera, by two types of antigens;

FIG. 2. is a graph showing the percent of matching between results obtained with two types of antigens; and

FIG. 3. is a graph demonstrating the difference in the performance of two antigen types emphasizing the superiority of the “new” (invention) over the previously home-made test performed by the inventor.

### DETAILED DESCRIPTION OF THE INVENTION

It has now been found that employing mycoplasma antigens obtained from the patients in a state of the disease yields superior immunoassays for detecting urogenital mycoplasmal infections, particularly if employing mycoplasma antigens from a mixture of samples of a plurality of patients. The invention provides a diagnostic method with surprising reliability and with a lowered number of false-positive results.

In the first stage, an ELISA type immunoassay was developed, for measuring the level of specific anti-mycoplasma antibodies in serum and in other body fluids comprising, for example, amniotic, synovial, cerebral, sperm, and tracheal lavage. The assay design included microtiter plates coated with mycoplasmal antigens prepared in our laboratory from the urogenital mycoplasma reference species obtained from international collections (ATCC). Then, the serum or any of the body fluids was incubated with the coated antigen followed by probing the antigen-antibody complexes formed, with anti-human IgG tagged with an enzyme (for example a commercially available peroxidase). Addition of a substrate to the complex resulted in a colored product, and its intensity was determined by spectrophotometry. In the second stage, this basic arrangement including plates or other matrix (for example inert particles or paper strips etc.) were coated with specific mycoplasma components originating from mycoplasma strains isolated from patients at the stage of their diseases. Thus, the material used for coating was representing those mycoplasma components that were expressed in disease and which were responsible for the pathogenic mechanisms. The patient's serum or other body fluids were incubated with the coated matrix and the antibodies in the specimen towards the mycoplasmal components were bound and their amount (e.g titer) was quantified. A vast number of human reference specimens were available in our laboratory (Soroka University Medical Centre, Beer-Sheva, Israel). The test development, was correlated with the clinical details regarding the involved patients, namely the pathological parameters, while including specimens from various diseases and disease stages. This approach enabled to evaluate the test reliability, sensitivity, reproducibility, and specificity; Furthermore, the comparison of the new test with other analytical techniques, such as culture and PCR, reassured the inventor that when using the mixed antigen preparation originating from a plurality of patients in acute stages of mycoplasma infection, a superior test emerged providing more reliable results, with less false positive values. Without wishing to be limited by any particular theory, the inventors believe that a mixture of antigens originating from a plurality of strains, and from a plurality of acutely infected patients, can better differentiate between non-symptomatic background mycoplasma populations and strongly pathogenic strains, reducing the false-positive results, and increasing sensitivity for detecting severe infections. The new test may incorporate other available immunoassay arrangements known in the art, such as procedural elements used in ELISA, in agglutination tests, absorption or precipitation tests, etc. While developing the test, the presence of antibodies in patients' blood or synovial fluids was correlated with the mycoplasma infection, and the prediction of further pathological developments was enabled (e.g. pregnancy complications, reactive arthritis, rheumatoid arthritis, urethritis, and other).

The present invention thus relates to a generally applicable immunoassay for practical detections of mycoplasma infections vs. colonization only. In one aspect of the invention, a method is provided for detecting those patients with mycoplasmal urogenital infections, that are at risk to develop a more sever disease that having an opportunistic mycoplasma colonization. In an important aspect of the invention, treating severe mycoplasmal disorders is enabled, comprising detecting the presence of a mycoplasmal causative agent followed by administering antibiotics.

In one very important aspect, the invention enables to develop and design immunoassays for use in diagnosing urogenital mycoplasma infections, precisely suiting the needs of certain environment, certain patient population, and certain laboratory equipment. When targeting a certain patient sector to be diagnosed, the specimens used in the preparation of mixed antigens will be collected from persons belonging to the same sector. The suitable mixed antigen will be employed in any immunoassay that is suitable for the available equipment.

In one embodiment of the invention, a mixed antigen is coupled to a matrix and serves for binding and visualizing anti-mycoplasma antibodies present in a tested body sample. Techniques known in the art enable quantifying and comparing antibody levels, for example by measuring color intensity. In a preferred application of an immunoassay according to the invention, the clinical laboratory collection builds a body of samples from patients with any relevant diagnostic and anamnestic combination. Utilizing this collection, a suitable mixed antigens is prepared for use in optimal immunoassays, for developing and calibrating the assays, for comparison with other available information and laboratory tests provided by independent methods, and for validating the resulting immunoassays.

In a preferred embodiment of the invention, a selected mixed antigen is bound to a piece of protein-binding matrix, and further is provided with color detection means, quickly revealing the presence of anti-mycoplasma antibodies characterizing a acute and/or chronic urogenital infection. Said piece of matrix may have the shape of a detection strip, and the detection means may comprise reagents for visualizing human antibodies, such as antihuman IgG-enzyme conjugate, enzyme substrate, etc., while the reagents may be compartmentalized in ampoules or otherwise, and prepared for the simple introduction into the visualizing process. Accompanied by a color scale and/or a usage leaflet, said piece of matrix and reagents provide a kit for detecting urogenital mycoplasmal infections.

The invention, thus, provides a superior laboratory test and commercial kit for detecting acute or chronic genitourinary infections caused by mycoplasmas in the situation where no general serological kit has been available in the market. The new test will significantly promote the analysis of mycoplasmal diseases. Beside a general use, a kit for detecting multiple urogenital mycoplasma is provided for distinguishing any desired combination of strains and species; based on the same methodology, strains belonging, for example, to Ureaplasma urealyticum, Ureaplasma parvum, Mycoplasma hominis, Mycoplasma fermentans, Mycoplasma genitalium, may be included.

The market is getting a reliable, simple, fast, easy and inexpensive kit with low false negative and low false positive results, complying with the needs of obstetrics and gynecology (high risk pregnancies, genetic amniocentesis, in-vitro fertilization, infertility), neonatalogy (intensive care, and others), urology (urinary tract infections in men and women), rheumatology (reactive arthritis, rheumatoid arthritis, and others), internal medicine, and STD clinics for high risk populations (sexually transmitted diseases).

The invention will be further described and illustrated in the following examples.

### EXAMPLES

### Example 1

A plurality of samples were taken from a plurality of patients suffering from acute or chronic urogenital infection. A mycoplasma strain was cultured from each sample, verifying the presence of mycoplasma by PCR. Six of the samples, in which proved mycoplasma belonged to either of Ureaplasma urealyticum and Ureaplasma parvum by means of PCR, were cultured in a 10B broth, containing 7.5% FCS (fetal calf serum). The cells were centrifuged at 27,000×g for 40 min and washed twice with phosphate buffer saline. The washed pellet of cells was sonicated in the presence of 1 mM phenylmethyulsulfonylfluoride (a proteases inhibitor), and the sonicate, containing all components of the cell, namely un fractionated mixture of proteins, lipoproteins and nucleic acids was used as the mycoplasma antigen.

### Example 2

A mixture of ureaplasmal antigens from six different strains of the ureaplasmas species (three Ureaplasma parvum (Up) and three Ureaplasma urealyticum (Uu)) was prepared. The strains were isolated from patients as described in Example 1, and is designated “New”. The identity of the species was verified by the PCR. Sera samples from 113 patients with different diseases, which had been previously analyzed in our laboratory, were tested with an ELISA employing the “New” mixed antigen. In parallel, ELISA was performed, employing antigens prepared from Ureaplasma parvum ATCC # 27815 formerly designated Ureaplasma urealyticum serotype 3 (Up), and Ureaplasma urealyticum ATCC # 27816 formerly designated Ureaplasma urealyticum serotype 4 (Uu). Each of them was used separately, and both are designated here as “Old”. These antigens were used for testing said 113 sera samples. Thus, ELISA tests based on three different antigen systems were compared.

Several types of sera were used, recorded in our laboratory as: a) Up positive, Uu positive, b) Up positive, Uu negative, c) Up negative, Uu positive, d) Up negative, Uu negative. Of all these sera, we included in the tests some sera that were: a) highly positive, b) positive, c) negative, d) intermediate positive, and e) borderline.

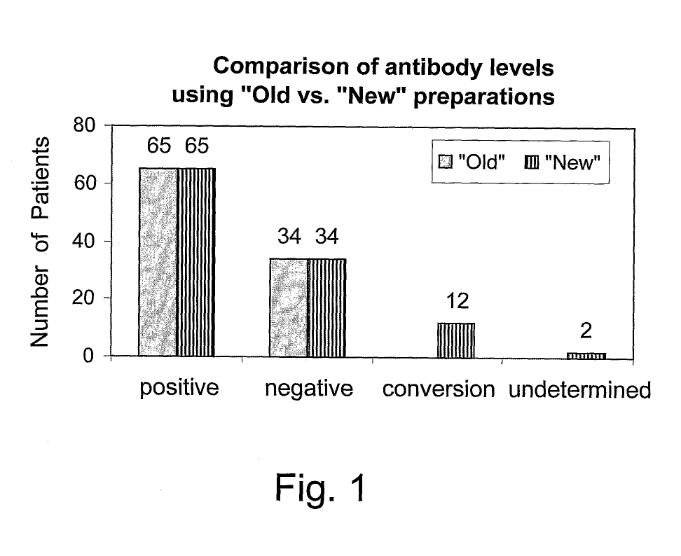

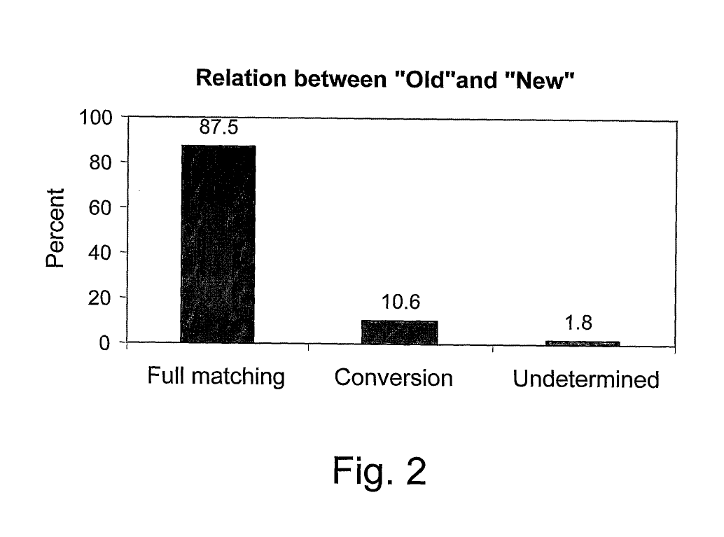

As seen in FIG. 1, all sera that were negative by the “Old” test were found negative by the “New”, and vice versa. Similarly, all sera that were highly positive by the “Old” test were also positive by the “New” one. Thus, in 87.5% of the sera, there was full agreement between the two tests (the“Old” and the “New”). As shown in FIG. 2. Only in 1.8% (2 patients out of 113) we could not determine the endpoint of the test. Moreover, 10.6% (12 out of 113) converted from positive (in the “Old”) to negative (with the “New”), suggesting that they were previously recorded as false-positive, and the “New” preparation is probably more reliable.

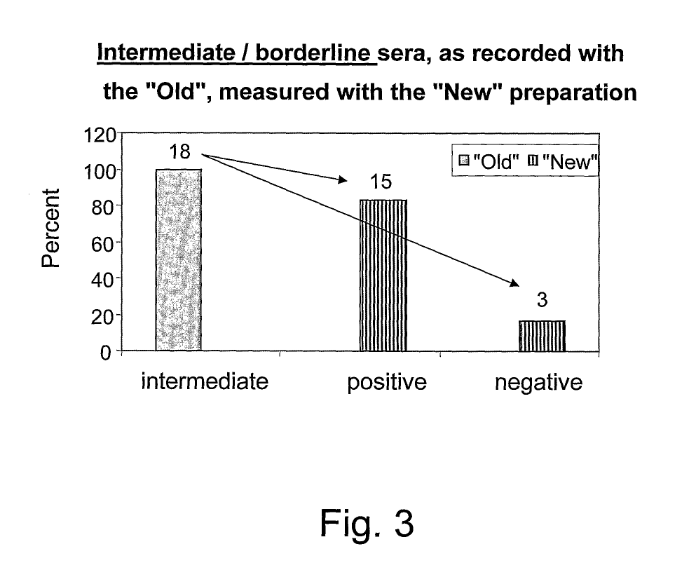

Another advantage of the “New” material was observed when we analyzed those sera that previously gave inconsistent results with the “Old” material and/or showed borderline levels of antibodies, which was reported as an “intermediate” result. As seen in FIG. 3, by using the “New” preparation, the “questionable” sera (n=18) were resolved, 83.3% being proven to be positive (15 out of 18 patients), and 16.7% became negative (3/15).

The test of the invention was proven to be much better and more decisive and reliable than the previously used test, performed with reference strains of Ureaplasmas spp. (Ureaplasma parvum and Ureaplasma urealyticum). In the above experiments, the sera were analyzed several times (2-4), with both “Old” and “New” tests, and the results on the “New” material were reproducible, except for two undetermined (FIG. 2). The new immunoassay method employing mixed mycoplasmal antigens detects specific anti-ureaplasma antibodies minimally as efficiently as the previous methods, and with less false results.

While this invention has been described in terms of some specific examples, many modifications and variations are possible. It is therefore understood that within the scope of the appended claims, the invention may be realized otherwise than as specifically described.

1. A method of detecting urogenital mycoplasmal infection in a biological sample taken from a patient to be diagnosed, comprising
i) providing a plurality of biological specimens from a plurality of pathological states, in patients suffering from mycoplasmal urogenital infections;
ii) preparing a whole cell preparation from said several mycoplasma strains obtained from patients;
iii) providing a plurality of mycoplasmal antigenic determinants (from each species) to be used as the material for detection of specific antibodies in the patients' sample;
iv) binding said mixed antigen on an immunoassay surface;
v) binding of the patients' biological sample with said surface of step iv, thereby binding of anti-mycoplasma antibodies eventually present in said sample onto said surface; and
vi) contacting the surface with a reagent for detecting human antibodies; thereby detecting an infection caused by a mycoplasmal agent from a wide spectrum of strains and species.

2. A method according to claim 1, wherein said plurality of antigens are prepared from at least six isolates, from 6 patients

3. A method according to claim 1, wherein said plurality of human strains preferably comprises at least six patients each infected with one or more mycoplasma species selected from the group consisting of Ureaplasma urealyticum, Ureaplasma parvum, Mycoplasma hominis, Mycoplasma fermentans, and Mycoplasma genitalium.

4. A method according to claim 1, wherein said preparing the proteinaceous composition comprises the preparation of bacterial sonicate by employing the steps of culturing, washing and rupture of concentrated mycoplasma cells.

5. A method according to claim 1, wherein said immunoassay surface comprises an item selected from well plate, polymer sheet, and paper strip.

6. A method according to claim 1, wherein said reagent comprises anti-human antibody conjugated with an enzyme and an enzyme substrate.

7. A mixed mycoplasmal antigen obtained as described in claim 1.

8. A method for the diagnosis of a genitourinary diseases associated with a mycoplasma infection in a human patient, comprising
i) providing a mixed antigen according to claim 7;
ii) examining a biological sample from said patient for the presence of an antibody against said mixed antigen and quantifying the level of said antibody; and
iii) comparing the level of said antibody in the patient's sample with standard samples of persons not suffering from acute mycoplasmal disease.

9. A kit for detecting urogenital infection in human caused by mycoplasmas, comprising
i) a mixed antigen according to claim 7 ? bound on a matrix;
ii) reagents for visualizing the presence of human antibodies; and
iii) instructions for use.

10. A kit according to claim 7, wherein said matrix is a strip comprising a protein binding polymer.

11. A kit according to claim 7, wherein said matrix comprises a sheet or well for detecting the presence of human anti-mycoplasma antibodies in more than one sample.

12. A kit according to claim 7, wherein said reagents is a conjugate of antihuman antibody or an enzyme substrate.

13. An antigen according to claim 7 for use in detecting a urogenital infections caused by a broad range of mycoplasmal antigens.

14. A method for following the success of treatment of a mycoplasmal urogenital infection in a patient in need of such treatment, comprising detecting the change in levels of antibodies to urogenital mycoplasma in said patient.
